(19) United States
(12) Patent Application Publication
(10) Pub. No.: US 2004/0116778 A1
(43) Pub. Date: Jun. 17, 2004

(54) Systems and methods for assessment of gross cultural assimilation

(76) Inventor: John Tyler Mauldin, LaGrange, GA (US)

Correspondence Address: Donald E. Hayes, Jr., 3097 Wembly Ridge, Atlanta, GA, 30340, US

(21) Appl. No.: 10/317,284

(22) Filed: Dec. 13, 2002

(51) Int. Cl. A61B 5/00

(52) U.S. Cl. 600/300

(57) ABSTRACT

The present invention is directed to systems and methods for performing psychological assessment directed at quantifying the patient's gross cultural assimilation factor. The present invention provides evaluation systems and methods that determines what life experiences a person has had, the impact of those experiences, and what behavioral outcomes resulted from the individual “making sense” of their experiences. The assessment is based on Dynamic Pattern Theory. This theory is defined using the key terms: 1. “dynamic”—any force that causes movement, and 2. “pattern”—a systematic action for negotiating life observed in behavior.

In combination, a dynamic pattern is a force that moves a developing person to create a systematic process for negotiating life experiences. Dynamic Pattern Theory is one explanation of the operational development of life patterns, and offers specific treatment interventions when patterns are not functioning adequately. Some embodiments may include components and/or steps that assist in clinical treatment.

A method of psychological assessment according to the present invention includes a variety of steps that may, in certain embodiments, be executed by the typical physiological-testing environment or is stored as computer executable instructions in and/or on any suitable combination of computer-readable media. A test subject will receive a test. The completed test is received and scored. The results of the test are provided to one or more designated healthcare providers associated with the test subject.

### REFERENCED APPLICATIONS

The present application is filed as a perfection of provisional Application No. 60/336,093 filed on Nov. 30, 2001.

### FIELD OF THE INVENTION

The present invention is directed to a novel and nonobvious approach to quantify, measure and evaluate a person at selected times during their progression through the inherent process of basic assimilation and adaptation to their culture. In general, the overall approach of identified as gross cultural assimilation. More specifically, without limitation, four basic psychological developmental problem areas that the person must solve to survive are addressed and are represented in the assimilation assessment by seven independent factors.

### BACKGROUND OF THE INVENTION

To formulate a theoretical framework for human behavior, one must begin with several a priori concepts. First, man is a biological being with a genetic structure. This genetic structure offers strengths and weaknesses, and gives individuals a basic starting point for their development. Biological strengths and weaknesses encompass two skill sets of varying degrees, A) cognitive (mental abilities) which includes, but is not limited to, thinking, reasoning, creativity, and interpretation of environmental and relational cues, and B) physiological (physical abilities) includes, but is not limited to, tactile, sensory, strength, endurance, agility, coordination, and provision of cues about our environment and relationships. Second, growth occurs in a natural progression from birth to death.

Third, development occurs as process of improving capabilities. For example, one may grow without walking but when he/she moves from crawling to walking development of balance, coordination has occurred. Development is generally an increase in an individual's ability and capability through learning.

Fourth, the application of cognitive abilities to life experiences creates an individual's belief structure and behavior patterns.

U.S. Pat. No. 5,435,324 to Brill discloses a method and apparatus for measuring a patient's psychotherapy progress is provided. Initial patient mental health is measured by administering a questionnaire measuring three psychological variables. The three psychological variable measures are combined into a mental health index. Following a number of sessions of psychotherapy, the patient's subsequent psychological condition is again measured using the three psychological variables. Patient session records are stored in a large database. Patient progress can thus be compared versus patient initial psychological condition; typical patient outcomes as stored in the database, and patient improvement as a function of a number of sessions of psychotherapy can be determined. The system further provides a case manager with a measure of the effectiveness of continued psychotherapy sessions, and a basis of comparison of various psychotherapy providers.

U.S. Pat. No. 4,895,518 to Arnold et al. discloses a system for assessing the diagnostic reasoning ability of learners engaged in a field of study employs a coded situation test and utilizes a computerized process for grading examinations taken by the learners and for rating their capacity for diagnostic reasoning. The computerized diagnostic reasoning evaluation instrument embodies storage in a test file of the situation test description that describes, illustratively in narrative form, a plurality of problems associated with the situation test employing a plurality of phrases. Each phrase has associated therewith a respective phrase identification symbol that is used by the examinee to record answers to the examination. In a nursing embodiment of the invention, each learner selects from a listing of possible problems. Those problems which are considered by the learner to be significant and present in the situation test are then identified by storing their respectively associated code symbol. In practice, the answers by the examinee may be entered directly into a computer by the examinee, or may be recorded by the examinee on paper and later entered into the grading computer by someone else. Subsequently, the learner is asked to identify which of the selected problems are significant using supporting data, and create from coded objectives, criteria, interventions, and rationale listings an intervention plan that is proposed by the learner to resolve the major problems. Grading is achieved by reference to a computerized answer key generated by an expert, such as a teacher, and stored in a computer memory as an answer key.

### SUMMARY OF THE INVENTION

The present invention sets forth and claims a novel and non-obvious system and method for assessing the gross cultural assimilation of a patient. In general, the invention discloses means for gathering data relating a patient's particular life experiences wherein at least a portion of said data relates to at least one of a predetermined list of psychological developmental problem areas, such as care, safety, boundaries and support. Additionally, the invention sets forth means for computing a single-valued quantity for each of said psychological developmental problem areas selected and thereby quantifiably representing the particular life experiences of that patient with respect to said psychological developmental problem area selected. Lastly, this invention provides means for comparing said single-valued quantity computed for each of said psychological developmental problem area selected with a predetermined benchmark quantity representing normative experiences for that psychological developmental problem area selected to obtain an indication of the patient's gross cultural assimilation with respect to said benchmark.

In accordance with the present invention, the present invention, the assessment of the cultural assimilation factor utilizes data regarding the patient's life experiences in a predetermined psychological developmental problem area that is gathered through a series of normative developmental questions. These questions are specifically designed to determine a range of interpersonal and environmental experiences that determine the social and cultural opportunities experienced by that individual patient.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention.

### BRIEF DESCRIPTION OF THE DRAWINGS AND APPENDIXES

The accompanying drawings and appendixes, which are incorporated in and constitute a part of this specification, illustrate embodiments of the invention and together with the description, serve to explain the principles of the invention.

FIG. 1 is a diagram of architecture for a typical system according to the present invention.

FIG. 2 is a diagram of architecture for another embodiment of the present invention.

FIG. 3 is a diagram of architecture for a further embodiment of the present invention.

Appendix A is a paper entitled “The Organization of Dynamic Pattern Assessment and Dynamic Pattern Theory.”

Appendix B is a sample collection of test questions for a patient age 12-19, including related subtests and subtest classifications in accordance with the present invention.

Appendix C is a Report Summary that identifies and defines various ranges within each of the seven factors assessed and quantified in accordance with the present invention.

Appendix D is a higher-level report summary that identifies and defines various ranges within the four psychological developmental problem areas that may be assessed and quantified in accordance with the present invention.

Appendix E is a sample of an actual report that may be used in accordance with the present invention to clearly and concisely convey the results of an assessment of a patient's gross cultural assimilation in accordance with the present invention.

### DETAILED DESCRIPTION OF THE PREFERRED EMBODIMENT

Before specifically addressing the particular features of the present invention, the next couple of paragraphs will address and clarify a few logistical issues related to the present invention.

First, while a complete and thorough explanation of the novel and non-obvious aspects and features of the present invention are set forth in the immediate body of the current document, additional supporting information is provided in the document entitled “The Organization of Dynamic Pattern Assessment and Dynamic Pattern Theory”, attached hereto and which is expressly incorporated by this reference herein. Hereinafter, this paper may be referred to as the Dynamic Pattern paper and accompanies the body of this text as Appendix A.

One or more preferred embodiments of the invention are now described in detail. Referring to the drawings, like numbers indicate like parts throughout the views. As used in the description herein, the meaning of “a,” “an,” and “the” includes plural reference unless the context clearly dictates otherwise. Also, as used in the description herein, the meaning of “in” includes “in” and “on” unless the context clearly dictates otherwise. The following terms shall have the meanings indicated except where the context clearly dictates otherwise:

Skill Set—A set of abilities innate within the genetics of an individual.

Cognitive—a mental capabilities skill set including but not limited to reasoning, judgment, problem solving, and creativity.

Physical—a set of abilities including, but not limited to, tactile, strength, endurance, agility, and coordination.

Growth—Normal physiological change occurring between birth and death.

Development—A sequential change process with individual based on life experiences.

Life Experiences—The sum total of good, bad, and ugly occurrences in an individual's life.

Environments—The places where life experiences occur.

Relationships—The people who participate in one's life experiences.

Stress—Any organism's non-specific response to any life experience.

Coping—Organized system for surviving stress.

Behavior Patterns—A systematic method for negotiating one's life experiences that can be observed in their behavior.

Lastly from a logistical standpoint, ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The specific novel and non-obvious features of the present invention will now be addressed. The initial discussion will focus more on the theoretical aspects of the present invention, followed by a description of one particular architectural embodiment that may be used to implement the present invention.

In general, the present invention is directed to a novel and nonobvious approach to quantify, measure and evaluate a person at selected times during their progression through the inherent process of basic assimilation and adaptation. In general, the overall approach of the present invention provides a tool for assessing/analyzing a psychological factor herein identified as gross cultural assimilation. More specifically, without limitation, four basic psychological developmental problem areas that the person must solve to survive are addressed and are represented in the assessment by seven independent factors.

The present invention provides a method and apparatus (i.e. system) to facilitate a reliable assessment and analysis of a person's life experiences, both environmental and interpersonal, which contribute to an individual's present personality organization. In accordance with the present invention, the experiences are identified and clustered into specifically selected impressed and expressed pattern factors. While these specific factors will be identified and discussed in greater detail below, in general, these factors reveal behavior patterns, assess developmental problems, and reliably predict future behaviors.

The phrase dynamic pattern theory will be used herein to reference the underlying theoretical premise of the present invention. In its broadest sense, dynamic pattern theory is based on the concept that observable behavior is the function of cognitive processes that are inevitably linked to the adaptive mechanisms of the individual. Following the adaptive process from Darwin, adaptation functions to pass genetic structure between generations. The genetic structure is passed as the structure of the most successful individuals in the culture. This cultural adaptation is the basis of individual assimilation into the culture, herein referred to as gross cultural assimilation.

The present invention proposes that the assimilation of an individual into the culture for successful competition is based on Darwin's natural selection and on his further theory of non-natural selection. Unnatural selection is the assistance of an individual within a system to survive competitively without the prerequisite skills to survive competitively. This interchange of selection between natural, which is dictated by the environment and relationships, is altered profoundly when the relationships can create or solve problem for the individual.

As used herein, the gross cultural assimilation factor is defined similarly to the intelligence factor “g.” It is a global construct demonstrated indirectly by the actions and sequential thinking, either logical or illogical, of the individual. It is idiosyncratic to its owner and represents his/her best interpretive effort at any particular developmental age. In accordance with the present invention, the gross cultural assimilation factor is quantifiable and measurable using specialized assessment tools designed for its evaluation and described and claimed herein. Lastly, the gross cultural assimilation factor organizes itself into self-fullfilling patterns and operates long term throughout life.

In order to develop an assessment approach that is truly reliable and practical, all appropriate information must be identified and ascertained. In accordance with the guidelines and basics of dynamic pattern theory, a personal questionnaire was designed that facilitates taking the information from merely a theoretical idea all the way to practical application in a manner that consistently produces an accurate individual experience pattern for a selected individual at that given time.

The basic tenets of the dynamic pattern theory are:

1.) Each person is born into an environment and develops (grows up) while passing though an increasingly broad range of environments;

2.) The original environment and each successive environment has people who will interact and affect the developing person;

3.) The developing person must learn from the environments and people within the environments what behaviors will work and what behaviors will not work to meet their needs;

4.) The developing individual suffers stress and must problem solve this stress to achieve successful need fulfillment;

5.) Any major stress that are outside the control of the individual must be problem solved with, without, or in spite of the people's help in each environment;

6.) The stress makes the environment and interaction fluid and dynamic requiring increasing specificity to problem solve;

7.) Each individual will produce increased specific solutions formulate behavior patterns that they understand and can apply over time as a solution to further stresses; and

8.) While the stresses and problem solving cannot be viewed directly it can be inferred from the observable behavior patterns and/or responses.

The tenets identified above are discussed in greater detail in the Dynamic Pattern paper attached hereto and as discussed earlier, is expressly incorporated by reference herein.

Another aspect of the present invention recognizes that this novel gross cultural assimilation assessment approach is devised with certain key principles in mind as required to be consistent with the tenets set forth above. Some of these key principles, as identified in accordance with the present invention, are briefly discussed below.

First, the assessment must reflect the developmental experiences, both environmentally and interpersonally, that create stresses for an individual. Secondly, the assessment must devise a cumulative strategy for assessing its expressed and impressed pattern factors. The assessment approach or system of the present invention demonstrates the degree of a given factor and whether that factor is present or absent. Lastly, the assessment interpretation must demonstrate the outcome effects of the expressed and impressed pattern factors on the experiencing individual.

Now that some general over-riding concerns and aspects of the present invention have been identified, more specifics of the exact mechanisms and embodiments used to implement the present invention will now be addressed. The first area addressed will focus on the manner in which the information needed to be known about, or received from; the person being evaluated is identified and gathered.

Question Design for Dynamic Pattern Assessment

Under existing techniques, some individuals use a subjective formulation regarding present psychological situations. However, such subjective formulations are insufficient to capture the range or depth of personality variables. Even where a Likert type scale allows the individual to rate their experience across levels of intensity, the outcome is merely the assessed measurement. Were an evaluation to end with this measurement, the results would suffice for general individual data about behavior. Questions designed from this approach are limited into singular behavioral rating issues. This singular issue approach denies the fluid nature or personality and discounts the value of developmental experience.

Measurements of this type do not determine the impact or functional value of a factor within the operating personality. While these existing measurements are subjective quantifications of a measured attribute structure existing for the rating individual, the measures are not functional in nature beyond the simple variable that each represents. The present invention proposes that questions designed from the dynamic pattern theory in accordance with the present invention can and do represent both the structure of the experience and the function of that experience within the operating personality.

Specifically, while the present invention respects the singular data gathering technique, the present invention advances it by using a novel combination of developmental experience and dynamic interpretation. To formulate questions with this fluid capacity, the present invention identifies the key developmental problems for a person passing through multiple environments and experiencing multiple relationships.

Four basic dynamic developmental problems that exist for all individuals are identified and used in accordance with the present invention. These four developmental problems are operational and occur in both relationships and environments.

Existing literature is replete with information about Reactive Attachment Disorder. This disorder appears to be a direct outgrowth of an infant or child not receiving proper interpersonal contact and attention during early developmental stages. An environmental example is Post Traumatic Stress Disorder. This disorder is the direct result of experiences in an unsafe environment where fear is a major and unrelieved stressor. Both interpersonal and environmental stresses create long term and pervasive developmental affects, producing difficult problems for the individual.

Each of the four problem areas is described in detail in the attached Dynamic Pattern theoretical paper. The four problem areas assessed in the Dynamic Pattern Assessment of the present invention are:

1.) Care—What specific type of care did the person receive?

2.) Safety—How safe did the person feel?

3.) Boundaries—What rules and rules consistency did the person experience?

4.) Support—What type of support system did the person experience?

All questions included in the invention are formulated to reflect environmental or interpersonal care, safety, boundaries, or support. Answers to these psychological developmental problems are the basis of appropriate questions in accordance with the assessment approach or system of the present invention. One acceptable set of questions is set forth in Appendix B, as the document entitled “Test Questions, Age 12-19.” It should be understood and appreciated that, while the present set of developmental questions presented herein is designed for specific ages, no one set of questions suffice for all ages. Therefore, each new developmental level may be best served by its own set of questions. Using the same theoretical approach, questions designed using this approach offer similar dynamic interpretive outcomes.

The next feature of the present invention was to determine what experiences (environment and relationship) are considered emotionally healthy and which are considered emotionally unhealthy. For the purpose of the present invention, the healthy emotional experiences were considered positive and desirable, while the emotionally unhealthy experiences were the converse. Developmentally it is considered a “good” occurrence to have a strong person in one's life. Conversely, not to have such a person is a “bad” occurrence from a developmental point of view. To determine the correct set of questions the invention organizes developmental experiences into a positive set of occurrences that could be scored either “Yes” or “No.” In each case, the “correct” answer benefited the examinee by adding a point to his/her score while the “incorrect” answer added nothing to the score. With this dichotomous scoring system, both true and false questions could be answered correctly by using a computerized program that allows for the assignment of “Yes” or “No” as the “correct” response.

For the purposes of the assessment, a clear picture is gained only if the experiences were either reported or not reported. Therefore, the assessment simply and directly asks whether a selected experience occurred or it did not. Thus, the individual taking the test reduces the subjective evaluation of degree and merely is asked to determine if the experience has occurred in his/her life. This strategy works because the basis of the questions is comprised on accepted normative developmental experiences.

The importance of the dichotomy is that the question has value to the outcome no matter what the response. In other words, not having an experience is just as clinically significant as having an experience. Thus, each question is important to the overall assessment of a person's personality organization.

Once a series of developmental questions were finalized, they were evaluated for wording and for content that reflected each of the four problem areas identified above, namely, care, safety, boundaries, and support. While the four problem areas are critical during question development, they raised the question whether the series of questions devised clustered into specific factors surrounding these problem areas. To discover if this belief was true, an independent factor analysis was completed. The factor analysis determined that within the four problem areas, the questions reflected seven independent factors. The present invention classified the factors either to be expressed on the person or impressed by the person. The difference lay in factors that come to the person verses factors that are offered by the person.

These seven independent factors are defined as follows:

1.) Modeling Experience—this area generally answers questions such as, “what were the overt and covert behaviors that an adult showed to the child during development.”

2.) Rules Experience—the area generally answers questions such as, “how consistent were the rules under which this child grew up during development.”

3.) Security Experience—this area generally answers questions such as, “what is the level of security experienced, not only interpersonal as with abuse but it includes physical accidents that may be unavoidable during development.”

4.) Support Experience—this area generally answers questions such as, “what was the support given to the developin g person as they struggled to become independent or overcome traumas during development. Is the help offered or is it unavailable?”

5.) Violation of Rules—this area generally answers questions such as, “how likely is the child to stay within the accepted boundaries for their age group?”

6.) Coping Skills—this area generally answers questions such as, “has the child developed sufficient skills to deal with the issues they experienced or are their coping skills undeveloped.”

7.) Relationship Give & Take—this area generally answers questions such as, “has the child matured and learned to cooperate in relationships, or are they struggling to get their way in all things.”

The factors of Modeling, Safety, Support, and Rules are factors that are “impressed on” the developing person by his/her environment and relationships. In contrast, the factors of Violation of Rules, Coping, and Relationship Give & Take are “impressed from,” the person onto the environment and relationships, i.e. “expressed on,” his/her environment and relationships by the developing person.

In accordance with the present invention, each of these factors is grouped into one of the four classifications of problem areas discussed earlier. Specifically, the core problem area is generally defined by the answers to questions focusing on factors of modeling experience and relationship give and take. The safety problem area is generally established based on answers and scores from the security experience and the coping skills assessment. Additionally, the boundaries problem area is generally defined by the answers to questions focusing on factors of rules experience and violation of rules. Lastly, the support problem area corresponds to the answers relating to the questions of the support experience.

Dynamic Pattern Assessment Scoring System

In accordance with the present invention, there are any number of mathematical options that may be used as the assessment scoring. One example is to simply by add the “correct” answers and divide by the overall number of questions for a selected factor. The percent calculated becomes the score for that factor. Because all questions reflect desirable developmental experiences, the higher the score, i.e. approaching 100%, the healthier the response set is considered. While this is true generally, as the score approaches 100%, it has been determined that extremely high percent scores 93%+are most often associated with idealized situations and relationships and reflect inaccurate representations. Conversely, scores below 7% are likewise considered skewed and represent attempts to demonstrate situations that are worse than actually experienced. Despite these scoring levels, the significance of the test is not diminished as will be explained in the interpretation section.

Other mathematical options that may be utilized in accordance with the present invention include a weighting of each of the professionally determined importance of each of the various independent factors. In other words, for certain persons being tested, it may be desirable to allocate more weight or importance to selected factors. Accordingly, each factor would not contribute one-seventh of the overall score but some factors more while some less.

One factor that may warrant adjustment of the particular mathematical weight assigned each factor could be the age of the person being tested. As a person grows older, from childhood, through adulthood and into older age, the number of that person's experiences that can be classified as “impressed from” that person to the environment and relationships and “impressed on” that person by the environment and relationships changes. Early in life, the environment and relationships affect the person more significantly than they do the environment and relationships. Contrarily, while an adult, the affect the person has on the environment and relationships increases. Lastly, in the later portion of one's life, certain physical and mental abilities diminish in a way that may require the person to be more reliant and thus affected by their environment and relationships as they were as children.

Statistical Analysis

Statistical analysis creates a knowledge base of ongoing information regarding the test effectiveness. Present shall sample reliability studies indicate a 0.90 reliability and validity is being evaluated. Further statistical analysis will be developed to determine successful from unsuccessful questions. This development will assist in improving or weighting the factor questions over time. Finally, overall change in a person's personality organization by factor will be determined.

Dynamic Pattern Assessment Delivery System

In the changing field of psychology, the Internet is becoming an increasingly common feature. In one embodiment of the present invention, the Dynamic Pattern Assessment is performed over the Internet. The test questions are contained in a secure, encrypted database that is firewall protected. The examinee performs the test in the presence of a licensed profession in the field of counseling or psychology. The questions are presented on a computer screen. Each questions is answered “true” or “false.” The Examinee controls the speed of administration based on his/her reading ability. If the examinee cannot read, the professional may read the test questions to him/her. In one particular embodiment of the present invention, the master test is six pages of 15 questions each, or 90 questions total. As stated earlier, the number of questions varies based on developmental age. As each page of questions is completed the Examinee clicks on a arrow in the lower right corner of the test question page. This click brings up the next page of questions. At the completion of the six pages, the Examinee submits the test for interpretation by clicking the submit button at the bottom of the last page.

Subtest Question Operation

While taking the master test in accordance with the present invention, certain questions are devised to require more information from the Examinee. The questions are clinical in nature and question non-developmental specific traumas, such as death, abuse, or victimization. These traumas are considered outside the normal developmental process but are important to the overall problem solving stress personality picture. In one embodiment shown herein, the Dynamic Pattern Assessment contains seventeen questions about non-developmental trauma. These questions required further information about the following traumas.

| | |
|---|---|
| Abuse Experience | Homicidal Impulses or Experience |
| Alcohol Experience | Legal Experience |
| Relationships Conflict Experience | Pain Experience |
| Coping Skills Experience | Sex Experience |
| Death Experiences | Suicide Impulses or Experience |
| Drugs Experience | Victim Experience |
| Employment Experience | School Experience |
| Finances Experience | |

Each of these experiences requires additional questions to specify what level of impact the experience has produces within the life of the Examinee. Additionally, each of these questions that trigger subtests is located on the master test and only activates the subtest if it is answered “incorrectly.”

Each subtest operates in the same mode as the master test once it is triggered. The screen changes to a “pop-up” of the subtest questions which when completed are submitted by clicking on a submit button at the bottom of the subtest screen. However, the subtests are scored slightly differently from the master test. Because of the desire to determine the impact of the trauma represented by the subtest, the number of “incorrect” questions is divided by the total questions score to determine the impact and seriousness of the trauma.

Dynamic Pattern Assessment Interpretation

One of the more unique and clinically helpful outcomes or aspects of the present invention is the scoring system. The concept of one embodiment involves determining the score as devised above and to present this score in ranges of 20 percentage points (0-19; 20-39; 60-59; 60-79; 80-100) with each range representing a level of personality organization. This original configuration has been used successfully, but will be replaced as data accumulates with a more specific range depicting the exact personality variable represented.

To specify the personality variables in each factor, the present invention takes several steps representing the tenets of the theory. Specifically, each independent factor was determined based on a continuum of positive to negative. The positive end of the scale has two levels—positive and very positive. Likewise, the negative end has negative and very negative. The middle of each factor is determined to be a psychological transition position with the individual having the capability to move either toward the positive or negative end of the scale.

Having established the continuum for each factor, the present invention utilizes present body of research to determine what the behaviors in each level of the continuum might be. This information was to be the observable behaviors both in interactions and in environments that would be present. For example, in modeling, what would be the worst potential role model and how would that model behave. In this specific case, it was determined that at the lowest level of modeling, the model was probably absent either emotionally through mental illness, drugs, or alcohol. This absence was not limited to environmental absence but included emotional and possible physical absence. Thus the relationships would be consistently absent of a model that could offer positive or substantive behaviors for the examinee to observe and emulate.

Once the general and specific attributes of each factor level were determined, it was speculated from the known literature what the impact of this specific behavior or lack of it might be developmentally on the examinee. This speculation was specific to the behaviors involved and researched for accuracy.

The attributes identified were coupled with the speculated impact organized into a set of outcome criteria for every factor. The results of this analysis are set forth in Appendix C entitled “Report Summary.” Additionally, in accordance with the present invention, a higher-level evaluation may also be performed if deemed beneficial by the professional administering the test. One example of such a report based on the four psychological developmental problem areas broadly is set forth in Appendix D.

As stated above, the factors of Modeling, Safety, Support, and Rules are factors that are impressed on the developing person by his/her environment and relationships. The factors of Violation of Rules, Coping, and Relationship Give & Take are impressed from, or expressed by, the person onto the environment and relationships.

As stated above, there are many mathematical options that may be used in accordance with the present invention to establish a numerical value for, and thus quantify, the gross cultural assimilation. This value is variable and based on the movement of the scores within the test as a person evolves and grows through life. It functions as an overall indicator of acculturation and can be used in assessment of improvement using the factors within the test.

Report Organization

The Dynamic Pattern Assessment reports the scores and outcome from the Examinee's test in several formats. A few of the acceptable versions are depicted in Appendix E, entitled “MPA Record Summary.” Specifically, the overall factors and corresponding percent scores are reported in a box format on the first page of the report. While below the score boxes, the Examinee's summary score is placed on a graph represent the unhealthy to healthy ranges of personality organization.

The interpretative ranges of the personality pattern are presented as clinical outcomes with limited recommendations for level of care. The trauma scores are present for each trauma triggered. These scores represent the risk in that clinical area and are accompanied with all the “incorrect” questions from the subtest to assist the clinician in understand the level of risk associated with that subtest.

Architecture of a Typical Environment

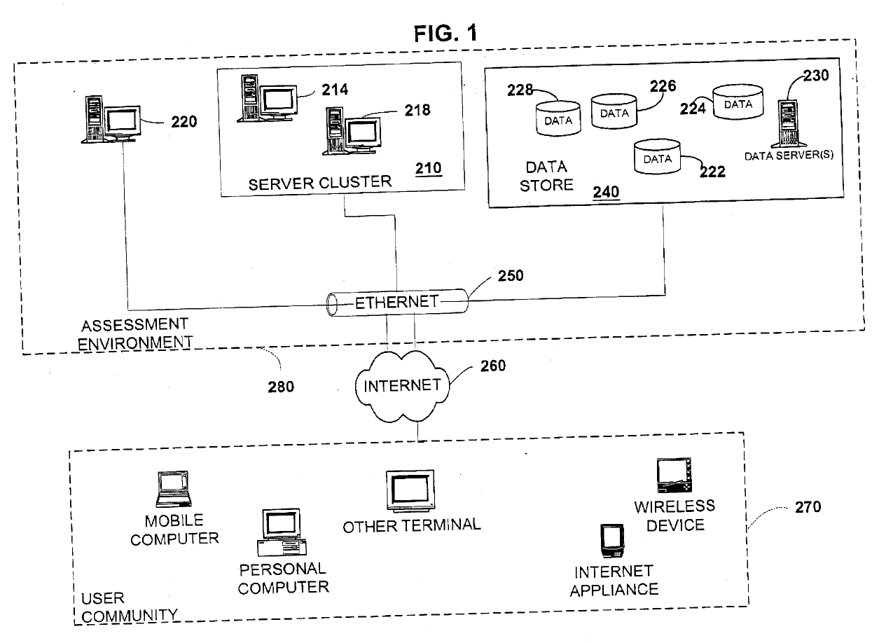

FIG. 1 depicts a typical environment according to the present invention. Members of the user community using suitable devices 270 can interact with the assessment environment 280 via a communications channel such as the Internet 260. A typical assessment environment 280 will include a cluster of servers 210 including one or more servers 214, 218 supporting psychological assessment functionality which, in some embodiments, may include test generation and transmission, tracking usage and report generation, as described more fully below. The assessment environment may include a separate system data store 240 for storing data associated with questions, test templates, test results, healthcare providers, subjects and other environment specific data. A data store 240, in a typical embodiment, may be implemented as a database system using one or more servers 230 with one or more internal or external data repository 222-228, as described more fully below.

The assessment environment 280 will also typically include a communication channel such as Ethernet 250 supporting communication among components of the environment 280. The assessment environment 280 may also optionally include one or more load-balancing devices 220, typically server systems, for distributing work among the components of the environment 280.

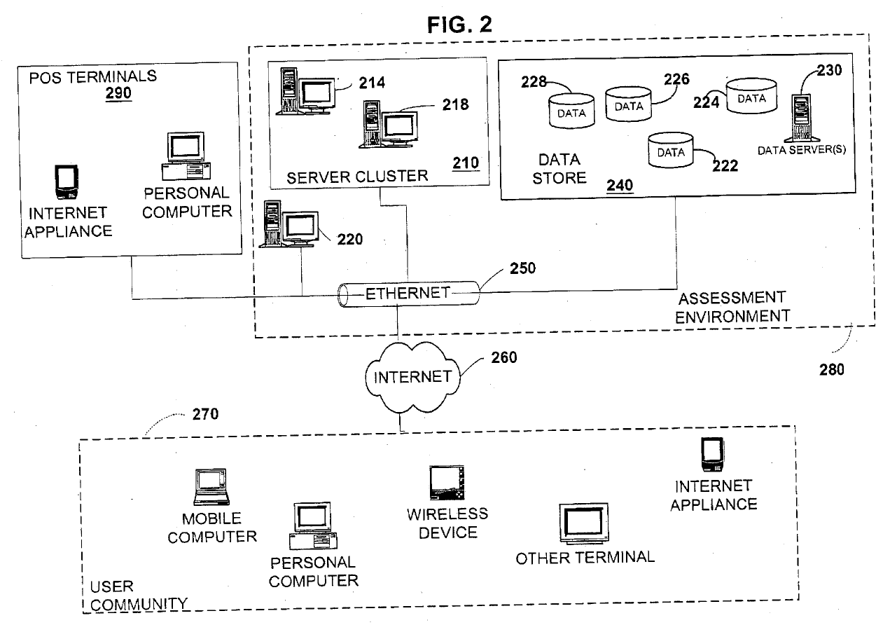

The assessment environment 280 may include a server cluster 210 of one or more servers (e.g. 214, 218) that provides environment functionality. These, or other servers (not shown), may support access to the environment by members of the user community 270, typically, environment administrators, healthcare providers, and test subjects. Access to the environment by these various users may be via any suitable communication channel, which in a typical embodiment will be a computer network such as the Internet 260 and/or Ethernet 250 (such as in the embodiment depicted in FIG. 2). In other environments, access may be via other forms of computer network, direct dial-up connection, dedicated connection, direct or indirect connection such as via a bus connection, parallel or serial connection, null modem connection or wireless connection utilizing an appropriate communication protocol such as BLUETOOTH, IRDA, 802.11b or other suitable channel as would be known to those skilled in the art. Some embodiments may use and/or require a combination of communication vehicles, such as those previously described, to serve as the communication channel. In some embodiments the access channel may provide security features, either session-based and/or layer-based; for instance, a secure socket layer (SSL) may be used with respect to an embodiment using the Internet 260 as the access communication channel. The one or more servers of the server cluster 210 may include or connect to a data store 240.

The conveyance of information to and from the user of the environment occurs via a link, or interface, to or with a suitable communication channel for conveying the information. The link will depend upon the environment implementation and the communication channel, or the first portion thereof where the communication channel is composed of several portions of potentially varying types. Depending upon the characteristic of the link, the link may allow selective communication with one or more user computers. In most cases, the environment communicates information to the user through a processor such as a computer, which may, in certain embodiments, provide server functionality, and be part of a server cluster; where the source of the communication is a processor, the link may be a wired or wireless modem, a serial or parallel interface, a network interface, a bus interface or combinations thereof where communication may occur via multiple communication channels or where differing types of communication occur through potentially different channels. The communication channel usually consists of one or more of the following types of channels: computer network, direct serial or parallel connection, dial-up connection, dedicated line connection, wireless connection, bus connection and combinations thereof. The communication channel may further consist of a variety of computer network types including an Ethernet, a token ring network, the Internet, and/or combinations thereof. Communication may use any suitable protocol; however, in most instances, the protocol selected will depend upon the communication channel. Typically, the protocol is one or more of the following protocols alone, or in combination where multiple types of channels form portions of the communication channel: HTTP, HTTPS, SMTP, FTP, GOPHER, and WAIS as layer 3 protocols and/or TCP/IP, IPX, NETBEUI, 802.11b, BLUETOOTH as layer 2 protocols and/or interprocess communication such as RPC, DCE, IIOP, COM, etc.

The various components of the environment 280 may communicate with each other through any suitable communication architecture including, but not limited to, a computer network such as a Ethernet 250, token ring network or the Internet 260; a direct connection such as a bus connection, parallel or serial connection, null modem connection, dedicated line or wireless connection utilizing an appropriate communication protocol such as BLUETOOTH; a dial-up connection; and appropriate combinations thereof. In embodiments where a single computer may provide all functional components of the environment, the communication may occur via bus connections, inter-process communication, shared files or some combination of these methods or other commonly used single-computer communication mechanisms.

The architecture, seen in FIG. 1, uses the Internet 260 and an Ethernet 250 as communication channels allowing access to the environment by various members of the user community 270. The environment uses a computer network such as the depicted Ethernet 250 to allow communication among the components of the environment; a router (not shown) may be included in the environment to manage such communication within the internal network as well as managing the interface between the internal network and the Internet 260. The functionality of the environment may be spread among a server cluster 210, a data store 240 and, in some embodiments, a load-balancing device 220. Where a load-balancing device 220 is present, the device may be responsible for allocating and managing distribution of access among various elements within the server cluster 210 and/or the data store 230. Users may access the environment through standard Web browser software or via specialized access software adapted for interfacing with the compliance environment 280.

The server cluster 210 provides the desired functionality of the environment 280. In some embodiments, the server cluster 210 may be divided into access servers and application servers where the access servers provide electronic access functionality such as by electronic mail server(s) and/or Web server(s) and the application servers provide the desired curriculum functionality. In some such embodiments, the one or more servers (e.g. 214, 218) in the server cluster 210 may be supported via Intel-compatible hardware platforms preferably using at least a PENTIUM III (Intel Corp., Santa Clara, Calif.) or UltraSPARC (Sun Microsystems, Palo Alto, Calif.) class processor. In some embodiments, assessment and/or access functionality, as further described below, may be distributed across multiple processing elements. The term processing element may be (1) a process running on a particular piece, or across particular pieces, of hardware, (2) a particular piece of processing hardware or (3) either as the context allows. The hardware platform would have an appropriate operating system such as WINDOWS/NT, WINDOWS 2000 or WINDOWS/XP Server (Microsoft, Redmond, Wash.), Solaris (Sun Microsystems, Palo Alto, Calif.), or LINUX (or other UNIX variant).

Depending upon the hardware/operating system platform, appropriate server software may be included to support the desired application, email, and Web server functionality. The Web server functionality may be provided via an Internet Information Server (Microsoft, Redmond, Wash.), an Apache HTTP Server (Apache Software Foundation, Forest Hill, Md.), an iPlanet Web Server (iPlanet E-Commerce Solutions—A Sun—Netscape Alliance, Mountain View, Calif.) or other suitable Web server platform. The email services may be supported via an Exchange Server (Microsoft, Redmond, Wash.), send mail or other suitable email server. Some embodiments may include one or more automated voice response (AVR) systems that are in addition to, or instead of, the aforementioned access servers. Such an AVR system could support a purely voice/telephone driven interface to the environment with hard copy output delivered electronically to suitable hard copy output device (e.g., printer, facsimile, etc.), and forwarded as necessary through regular mail, courier, inter-office mail, facsimile or other suitable forwarding approach.

Application servers in some embodiments may be iPlanet Application Servers (iPlanet E-Commerce Solutions—A Sun—Netscape Alliance, Mountain View, Calif.), WebSphere Servers (International Business Machines, Armonk, N.Y.), Tomcat Java Servlet/JSP Engine (Apache Software Foundation, Forest Hill, Md.), or WebLogic Application Server (BEA Systems, Inc., San Jose, Calif.). In some embodiments, the application services may be provided through programmed pages on the Web server; such pages may use ASP (Microsoft, Redmond, Wash.), ColdFusion (Macromedia, San Francisco, Calif.), Java Servlet (Sun Microsystems, Palo Alto, Calif.) or other suitable technologies to provide server side logic and may use ActiveX (Microsoft, Redmond, Wash.), VBScript (Microsoft, Redmond, Wash.), JavaScript/ECMAScript (ECMA, Geneva, Switzerland) or other suitable technologies to support client side logic.

The data store 240 provides for the storage and, potentially, the management of the data required by the environment. A typical data store 240 will include one or more storage devices (e.g. 222, 224, 226 & 228), and in some embodiments, may include one or more data servers 230 to receive and service data requests. The data store depicted in FIGS. 1-3 uses a server 230 and several external data repository 222, 224, 226 & 228. These depictions are representative only, and consequently, other data store architectures may have single, multiple and/or varied servers and storage elements. For example, the system data store may use internal storage devices connected to one or more of the server processors (214, 218) of the server cluster 210. In embodiments where a single processor supports all functionality of the environment, a local hard disk drive may serve as the system data store, and a disk operating system executing on the single processor acting as a data server may support receive and service data requests.

Information concerning different users (including administrators, question developers, healthcare providers, test subjects, etc.), healthcare facilities using the environment, question content, test templates content, and records tracking environment usage and test results may be stored in the data store 240. The various types of content discussed above may be available in any suitable format such as HTML, XML, SGML, PDF (Adobe Systems Inc., San Jose, Calif.), VRML, PostScript, Encapsulated Postscript, text, TIFF, GIF, CFM (Cold Fusion), ASP (Active Server Pages), etc. and may include other embedded active components such as HTML forms, JavaScript routines, Java applets, etc.

It will be understood by those of skill in the art that these different types of information may be logically or physically segregated within a single system data store; multiple related data stores accessible through a unified management system, which together serve as the system data store; or multiple independent data stores individually accessible through disparate management systems, which may in some embodiments be collectively viewed as the system data store. The various storage elements that comprise the physical architecture of the system data store may be centrally located, or distributed across a variety of diverse locations.

The architecture of the data store 240 may vary significantly in different embodiments. In several embodiments, database(s) are used to store and manipulate the data; in some such embodiment, one or more relational database management systems, such as DB2 (IBM, White Plains, N.Y.), SQL Server (Microsoft, Redmond, Wash.), ACCESS (Microsoft, Redmond, Wash.), ORACLE 8i (Oracle Corp., Redwood Shores, Calif.), Ingres (Computer Associates, Islandia, N.Y.), or Adaptive Server Enterprise (Sybase Inc., Emeryville, Calif.), in connection with a variety of storage devices/file servers that may include, in some embodiments, an tape library such as Exabyte X80 (Exabyte Corporation, Boulder, Colo.), a storage attached network (SAN) solution such as available from (EMC, Inc., Hopkinton, Mass.), a network attached storage (NAS) solution such as a NetApp Filer 740 (Network Appliances, Sunnyvale, Calif.), or combinations thereof. In other embodiments, the data store may use database systems with other architectures such as object-oriented, spatial, object-relational, or hierarchical or may use other storage implementations such as hash tables or flat files or combinations of such architectures. Such alternative approaches may use data servers other than database management systems such as a hash table look-up server, procedure and/or process and/or a flat file retrieval server, procedure and/or process.

Following is a typical database design for an embodiment that utilizes a relational database management system such as ACCESS or SQL Server.

| | |
|---|---|
| Users | Address |
| Id | Phone (3): house, cell, work |
| Password | Age |
| Billing address | Answer |
| Pager | Link code- |
| E-mail (2): home, work | Link question to question |
| Sex | Associated number |
| Martial status | Comments- |
| Spouse Spouse address | Type of question (feeling, coping)- |
| Legal Guardian | Date time entered- |
| Emergency Contact | Entered by |
| Emergency contact address | User scores- |
| Emergency contact e-mail | Id- |
| Hospital (address) | Event- |
| Doctor (address) | Question- |
| Address | Answer- |
| Comments | Score- |
| Billing status | Date time entered- |
| Method of payment | Score Bank Id |
| Hospital Code Doctor Code | Score Bank- |
| Test Bank | Id- |
| Event | Event- |
| Event group | Event Group- |
| Question | Recommended Treatment(memo)- |
| Question order of appearance | Score Range |

User Tables

The document entitled “PsyPractice Assessment Center—Administrative Guide”, attached hereto and expressly incorporated by this reference herein, provides greater detail regarding embodiments of the present invention.

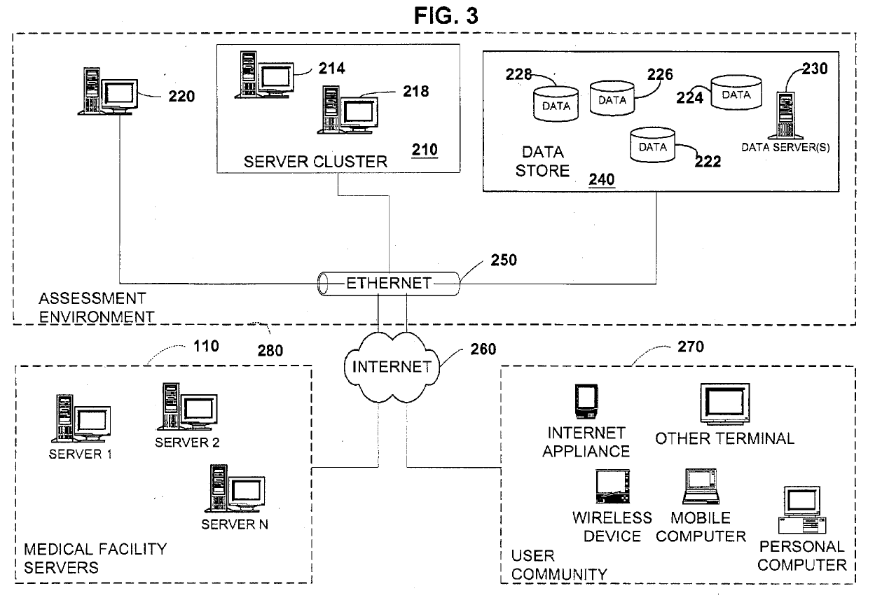

FIGS. 1-3 depict exemplary system embodiments of the present invention. In FIG. 1, the assessment environment is provided through a centralized provider. All users 270 access this provider via a communications channel such as the Internet 260 as depicted. In FIG. 2, the assessment environment 260 is locally installed and managed at a health care point of service (POS) provider. The system may be accessed via an internal communication channel such as the depicted Ethernet 250 by users within the POS facility 290. The environment 260 may be further accessed via an external communication channel such as the Internet 260 by other users 270. In another embodiment similar to that of FIG. 2, no external communication channel is used and sole access is provided to users within the POS facility. The embodiment depicted in FIG. 3 is similar to that of FIG. 1; however, in this embodiment, medical facility servers 110 may be accessed via a communications channel. The medical facility servers 110 may be queried for information regarding various users, including test subjects and healthcare providers, of the environment 250. The communication channel depicted is the Internet 260 but in some embodiments may be an internal channel such as an Ethernet or other local area network.

Various methods and functions as exhibited in various embodiments according to the present invention are described below with respect to assessment functionality including test generation and transmission, tracking usage and report generation. In some embodiments, one or more processors within architectures of the environments as described above may execute the steps in such methods and provide such functionality. The functionality may spread across multiple processing elements; in certain embodiments, these processing elements may logically and/or physically be divided into access, assessment logic, and data storage processing elements where functionality is allocated appropriately among such processing elements. In other embodiments, any suitable computer readable storage device, including primary storage such as RAM, ROM, cache memory, etc. or secondary storage such as magnetic media including fixed and removable disks and tapes; optical media including fixed and removable disks whether read-only or read-write; paper media including punch cards and paper tape; or other secondary storage as would be known to those skilled in the art, may store instruction that upon execution by one or more processors cause the one or more processors to execute the steps in such methods and to provide such functionality.

Psychological Assessment Processes

The test subject receives a test formulated to ask general questions about life experiences. It offers a series of questions that reflect developmental life experiences in both positive and negative statements. For each question, the answer is clear regarding the correct developmental response. The test author has designated the correct responses as life experiences producing maximum physical and emotional health for a developing person. Other items reflect stressors and stressful life events. These items are included to determine if the person has experienced events that would reduce their developmental success in environments or relationships. A set of coping questions offers information regarding the individual's coping abilities. The scoring determines problem solving skill sets both environmentally and interpersonally. It examines early historical events to determine trauma and to evaluate overall sense of care, support, safety, and boundary development.

To ask questions that would delineate the above information, it became necessary to use both established testing ideology (mental status) and to develop a new paradigm. The standard mental status questionnaires in the industry are roughly equivalent. The questions about life experiences are derived by taking all the environments that a developing person might experience (history) and determining what relationships (people) are available in those environments. For example in the birth home environment, interactions might occur with parent(s), siblings, or relatives.

The questions encompass the areas discussed above, and in the attached paper. The computerized test format allows a question in the general test to trigger subtest questions. For example, a general question about abuse would trigger a pop-up subtest of abuse questions that determine severity, intensity, and duration.

Each major area and subtest score is organized into five levels based on percent correct score. The major findings are discussed in detail in the outcome report and the deviant subtest questions are present individually for clinician review.

The report is intended to offer a concise starting point for treatment. It reduces intake time and allows a therapist to begin treatment quickly. The information offers basics about individual patterns and the concise nature of a problem. Acting as a springboard for therapy, this is a fluid/dynamic assessments tool that blends with most treatment modalities. It utilizes the unique approaches of Dynamic Pattern Therapy, but is not dependent on this particular theory to make it a useful diagnostic tool. The general concepts of Dynamic Pattern Therapy are described in the attached Dynamic Pattern paper.

The employed process of administering the test is described in greater detail in the attached document entitled “PsyPractice Assessment Center—Administrative Guide.” Test scoring, and alternative approaches, is discussed in more detail in the attached paper Dynamic Pattern paper. The Dynamic Pattern paper further describes various embodiments of the present invention that include clinical use of the computerized test results.

Throughout this application, various publications may have been referenced. Also, in the attachment entitled “The Organization of Dynamic Pattern Assessment and Dynamic Pattern Theory” references a variety of publications listed in the section entitled References. The disclosures of these publications in their entireties are hereby incorporated by reference into this application in order to more fully describe the state of the art to which this invention pertains.

Closing

The Predictable Assessment of Socio-Cultural Personality Patterns

Historically, it has been difficult to predict human behavior. The present invention devises an assessment procedure for personality patterns by assessing historical experiences and determining the cause/effect on the personality. The assessment is designed to give evidence that accumulated information produces statistics and inference about predictable behaviors, outcomes and expectations for future behaviors.

The present invention proports that:

1. Human behavior is rational and irrational.

2. Human behavior is a function of interpersonal and environmental experiences.

3. The interpersonal and environmental experiences are developmental.

4. The person who has the experience interprets these developmental experiences.

5. The force of the experience is a dynamic force, which cannot be witnessed directly but can be inferred indirectly from other evidence. The present test performs the indirect analysis for the inferred dynamic.

6. Human behavior is systematic.

7. Humans construct social systems surrounding their behavior and beliefs.

8. The breakdown of social systems to individual systems requires assessing normative experiences that occur within the social experiences and cultural structure.

9. The present assessment creates a set of normative developmental questions that determine a range of interpersonal and environmental experiences that determine the social and cultural opportunities experienced by an individual.

10. The stages of the experiences are defined by known developmental theory.

11. The assessment is delivered using the web and personal computer.

12. The parameters of learned behavior are along certain specific parameters.

13. The parameters are Care, Safety, Boundaries, and Support. The assessment of these factors follow both interpersonal and environmental experiences.

14. Best indicator of future behavior is past behavior. Therefore, past treatment sets the stage for future behavior.

15. The present assessment determines present functioning from past socio-cultural experiences.

16. The assess the past to predict future behaviors.

17. Behaviors can be adaptive or maladaptive to the environment and relationships.

18. Know the degree of adaptation or maladaptation of an individual assists in treatment recommendations.

19. Knowing the degree of adaptation or maladaptation gives an assessment process for progress in any known form of treatment and improvement of socio-cultural adaptation.

In contrast to existing prior systems and theoretical comments above, the present invention offers the following:

1. Web enabled application.

2. Automatic interpretation test results.

3. Reported test results without interpretation.

4. Computerized pretest/posttest comparison.

5. Pop up subtests to gather specific functioning data from main test questions.

6. Secure database encryption.

7. Three level password security system.

8. Single administration access to specific user records.

9. Online ability to add questions, subtests and tests as needed.

10. Online ability to edit, change or eliminate questions.

11. Online ability to have questions active, inactive, or experimental.

12. Online user test taking.

13. Ability to create general survey questions and deliver user survey results.

It will be understood that the embodiments described above are merely illustrative of the principles of this invention. It will be readily appreciated by those skilled in the art that many deviations and various modifications may be made from the specific embodiments disclosed in this specification without departing from the scope and spirit of the invention.

What is claimed is:

1. A system for assessing the gross cultural assimilation of a patient comprising:
means for gathering data relating a patient's particular life experiences wherein at least a portion of said data relates to at least one of a predetermined list of psychological developmental problem areas;
means for computing a single-valued quantity for each of said psychological developmental problem areas selected and thereby quantifiably representing the particular life experiences of that patient with respect to said psychological developmental problem area selected; and
means for comparing said single-valued quantity computed for each of said psychological developmental problem area selected with a predetermined benchmark quantity representing normative experiences for that psychological developmental problem area selected to obtain an indication of the patient's gross cultural assimilation with respect to said benchmark.

2. The cultural assimilation assessment system of claim 1 wherein said data regarding the patient's life experiences in a predetermined psychological developmental problem area is gathered through a series of normative developmental questions that determine a range of interpersonal and environmental experiences that determine the social and cultural opportunities experienced by that individual patient.

3. The cultural assimilation assessment system of claim 2 wherein said predetermined developmental psychological developmental problem areas are selected from the group consisting of care, safety, boundary, and support.

4. The cultural assimilation assessment system of claim 3 wherein said data regarding the patient's life experiences in the care psychological developmental problem area is gathered through a series of normative developmental questions that are directed toward factors of modeling experiences and relationship give and take experiences.

5. The cultural assimilation assessment system of claim 3 wherein said data regarding the patient's life experiences in the safety psychological developmental problem area is gathered through a series of normative developmental questions that are directed toward factors of security experiences and coping skills.

6. The cultural assimilation assessment system of claim 3 wherein said data regarding the patient's life experiences in the boundaries psychological developmental problem area is gathered through a series of normative developmental questions that are directed toward factors of rules experiences and violation of rules experiences.

7. The cultural assimilation assessment-system of claim 3 wherein said data regarding the patient's life experiences in the support psychological developmental problem area is gathered through a series of normative developmental questions that are directed toward factors of support experiences.

8. The cultural assimilation assessment system of claim 1 wherein the single-valued quantity computed for a selected psychological developmental problem area is mathematically combined with the single-valued quantity computed for a different psychological developmental problem area to generate a numeric indication of the patient's gross cultural assimilation factor.

9. The cultural assimilation assessment system of claim 8 wherein the single-valued quantities computed for each selected psychological developmental problem area are mathematically combined equally with each of the other single-valued quantities computed for other psychological developmental problem area to generate the numeric indication of the patient's gross cultural assimilation factor.

10. The cultural assimilation assessment system of claim 8 wherein the single-valued quantities computed for at least one of the selected psychological developmental problem areas is mathematically weighted relative to the other single-valued quantities computed for other psychological developmental problem area prior to being mathematically combined to generate the numeric indication of the patient's gross cultural assimilation factor.

11. A method for assessing the gross cultural assimilation of a patient comprising the steps of:
gathering data relating a patient's particular life experiences wherein at least a portion of said data relates to at least one of a predetermined list of psychological developmental problem areas;
computing a single-valued quantity for each of said psychological developmental problem areas selected and thereby quantifiably representing the particular life experiences of that patient with respect to said psychological developmental problem area selected; and
comparing said single-valued quantity computed for each of said psychological developmental problem area selected with a predetermined benchmark quantity representing normative experiences for that psychological developmental problem area selected to obtain an indication of the patient's gross cultural assimilation with respect to said benchmark.

12. The cultural assimilation assessment method of claim 11 wherein said data regarding the patient's life experiences in a predetermined psychological developmental problem area is gathered through a series of normative developmental questions that determine a range of interpersonal and environmental experiences that determine the social and cultural opportunities experienced by that individual patient.

13. The cultural assimilation assessment method of claim 12 wherein said predetermined psychological developmental problem areas are selected from the group consisting of care, safety, boundary, and support.

14. The cultural assimilation assessment method of claim 13 wherein said data regarding the patient's life experiences in the care psychological developmental problem area is gathered through a series of normative developmental questions that are directed toward factors of modeling experiences and relationship give and take experiences.

15. The cultural assimilation assessment method of claim 13 wherein said data regarding the patient's life experiences in the safety psychological developmental problem area is gathered through a series of normative developmental questions that are directed toward factors of security experiences and coping skills.

16. The cultural assimilation assessment method of claim 13 wherein said data regarding the patient's life experiences in the boundaries psychological developmental problem area is gathered through a series of normative developmental questions that are directed toward factors of rules experiences and violation of rules experiences.

17. The cultural assimilation assessment method of claim 13 wherein said data regarding the patient's life experiences in the support psychological developmental problem area is gathered through a series of normative developmental questions that are directed toward factors of support experiences.

18. The cultural assimilation assessment method of claim 11 wherein the single-valued quantity computed for a selected psychological developmental problem area is mathematically combined with the single-valued quantity computed for a different psychological developmental problem area to generate a numeric indication of the patient's gross cultural assimilation factor.

19. The cultural assimilation assessment method of claim 18 wherein the single-valued quantities computed for each selected psychological developmental problem area are mathematically combined equally with each of the other single-valued quantities computed for other psychological developmental problem area to generate the numeric indication of the patient's gross cultural assimilation factor.

20. The cultural assimilation assessment method of claim 18 wherein the single-valued quantities computed for at least one of the selected psychological developmental problem areas is mathematically weighted relative to the other single-valued quantities computed for other psychological developmental problem area prior to being mathematically combined to generate the numeric indication of the patient's gross cultural assimilation factor.
